(19) United States
(12) Patent Application Publication
(10) Pub. No.: US 2013/0158080 A1
(43) Pub. Date: Jun. 20, 2013

(54) METHODS FOR TREATING HYPERTENSION IN BLACK PATIENTS

(71) Applicant: Takeda Pharmaceuticals U.S.A., Inc., Deerfield, IL (US)

(72) Inventor: Stuart Kupfer, Deerfield, IL (US)

(73) Assignee: Takeda Pharmaceuticals U.S.A., Inc., Deerfield, IL (US)

(21) Appl. No.: 13/709,853

(22) Filed: Dec. 10, 2012

Related U.S. Application Data
(60) Provisional application No. 61/570,965, filed on Dec. 15, 2011.

(51) Int. Cl. A61K 31/4245; A61K 31/4035

(52) U.S. Cl. CPC A61K 31/4245; A61K 31/4035
USPC 514/364

(57) ABSTRACT

The present invention relates to methods of treating hypertension in a black patient using a therapeutically effective amount of a combination of an angiotensin II receptor blocker and chlorthalidone.

### CROSS-REFERENCE TO RELATED APPLICATION

This claims the benefit of U.S. Provisional Application No. 61/570,965 filed on Dec. 15, 2011, the contents of which are herein fully incorporated by reference.

### FIELD OF THE INVENTION

The present invention relates to methods of treating hypertension and other cardiovascular or renal diseases in black patients. More specifically, the present invention relates to methods of treating hypertension in a black patient using a therapeutically effective amount of a combination of an angiotensin II receptor blocker and chlorthalidone.

### BACKGROUND

Blood pressure (“BP”) is defined by a number of haemodynamic parameters taken either in isolation or in combination. Systolic blood pressure (“SBP”) is the peak pressure exerted on the walls of the arteries during the contraction phase of the ventricles of the heart. Diastolic blood pressure (“DBP”) is the minimum pressure exerted on the vessel walls when the heart muscle relaxes between beats and is filling with blood. The mean arterial blood pressure is the product of cardiac out put and peripheral vascular resistance.

Pre-hypertension has been defined as a SBP in the range of from 120 mmHg to 139 mmHG and/or a DBP in the range of from 80 mmHg to 89 mmHg. Pre-hypertension is considered to be a precursor of hypertension and a predictor of excessive cardiovascular risk (Julius, S., et al., N. Engl. J. Med., 354:1685-1697 (2006)).

Hypertension, or elevated BP, has been defined as a SBP of at least 140 mmHg and/or a DBP of at least 90 mmHg. By this definition, the prevalence of hypertension in developed countries is about 20% of the adult population, rising to about 60-70% of those aged 60 or more, although a significant fraction of these hypertensive subjects have normal BP when this is measured in a non-clinical setting. Hypertension in individuals with diabetes or renal impairment has been defined as a SBP of at least 130 mmHg and/or a DBP of at least 80 mmHg. Some 60% of this older hypertensive population have isolated systolic hypertension, i.e. they have an elevated SBP and a normal DBP. Hypertension is associated with an increased risk of cardiovascular death, stroke, myocardial infarction, atrial fibrillation, heart failure, peripheral vascular disease and renal impairment (Fagard, R. H., Am. J. Geriatric Cardiology, 11 (1), 23-28 (2002); Brown, M J and Haycock, S; Drugs, 59 (Suppl 2), 1-12 (2000)).

The renin-angiotensin system (“RAS”) is a group of related hormones that act together to regulate blood pressure. The RAS is referred to as a “system” because each part influences the other parts and all are necessary for the whole to function correctly. The RAS, working together with the kidneys, is the body's most important long-term blood pressure regulation system. While short-term blood pressure changes can be caused by a variety of factors, almost all long-term blood pressure adjustments are the responsibility of the kidneys and the renin-angiotensin system.

When the blood pressure drops for any reason, certain cells in the kidney detect the change and release renin into the blood stream. By itself, renin does not really affect the blood pressure. Instead, it floats around and converts inactive forms of angiotensin into angiotensin I. The inactive forms of angiotensin, which are produced by the liver, are not able to alter the blood pressure until renin changes them into angiotensin I.

Angiotensin I is able to alter the blood pressure to some degree, but it is not potent enough to cause large changes. Rather, most angiotensin I is converted to angiotensin II, a much more potent hormone that causes large changes in blood pressure. This second conversion happens mainly in the lungs as a result of an angiotensin converting enzyme (ACE).

Angiotensin II can act directly on blood vessels to cause blood pressure increases. It also stimulates the release of aldosterone. Aldosterone is a potent vasoconstrictor that causes large increases in blood pressure, but can also change the baseline filtering activity of the kidneys. Aldosterone causes the kidneys to retain both salt and water, which, over time, increases the amount of water in the body. This increase in the amount of water raises blood pressure.

Hypertension in blacks is a major clinical and public health problem because of the high prevalence and premature onset of elevated BP as well as the high burden of co-morbid factors that lead to pharmacological treatment resistance (such as obesity, diabetes mellitus, depressed glomerular filtration rate, and albuminuria). Classically, blacks have been considered to be a “low-renin” race. Specifically, it is known in the art that 30% of blacks with hypertension have no detectable plasma renin activity. The etiology of black hypertension is multifactorial. In addition to the RAS, reports have implicated abnormalities in the sympathetic nervous system, kallikrein-kinin system, nutritional elements including low levels of potassium or calcium intake, as well as a host of other economic, behavioral and psychosocial factors.

Angiotensin II receptor blockers (ARB) specifically antagonize or block the action of angiotensin II type 1 receptors. This results in an inhibition of the physiological action of angiotensin II. A number of ARBs are known in the art. Examples include, valsartan, losartan, candesartan, eprosartan, irbesartan, olmesartan, tasosartan, telmisartan, azilsartan medoxomil, etc.

Thiazide-type diuretics, which includes chlorthalidone and hydrochlorothiazide, have been effectively used in long-term monotherapy to lower blood pressure, enhance the efficacy of other antihypertensive agents, and reduce cardiovascular events. (Ernst, M., et al., N. Engl. J. Med., 361:2153-2164 (2009)).

Studies have shown that the combination of an ARB and a diuretic achieved greater blood pressure reductions than either agent alone, with no significant increase in adverse effects. A number of fixed-dose combinations of an ARB and a thiazide-type diuretic are known. For example, Benicar HCT is a fixed-dose combination of olemsartan medoxomil plus hydrochlorothiazide. Benicar HCT is commercially available as tablets containing 20 mg or 40 mg of olmesartan medoxomil combined with 12.5 mg of hydrochlorothiazide, or 40 mg of olmesartan medoxomil combined with 25 mg of hydrochlorothiazide. Diovan HCT is a fixed-dose combination of valsartan and hydrochlorothiazide. Diovan HCT is commercially available as tablets containing 160 mg of valsartan combined with 12.5 mg of hydrochlorothiazide or 320 mg of valsartan combined with 25.0 mg of hydrochlorothiazide. Tekturna HCT is a fixed-dose combination of aliskiren plus hydrochlorothiazide. Tekturna HCT is commercially available as tablets containing 150 mg of aliskiren combined with 12.5 mg of hydrochlorothiazide, 150 mg of aliskiren combined with 25.0 mg of hydrochlorothiazide, 300 mg of aliskiren combined with 12.5 mg of hydrochlorothiazide or 300 mg of aliskiren combined with 25.0 mg of hydrochlorothiazide.

It is known in the art that some antihypertensive drugs have smaller blood pressure effects (as monotherapy) in black patients. In other words, the blood pressure effect of the drug is different (namely, less) in black patients than white patients. It is also known in the art that fixed-dose combinations of an ARB and thiazide-type diuretic produce unpredictable results in black patients. For example, the label for Benicar HCT states that the “effect [of the combination] was smaller in black patients (usually a low-renin population), as has been seen with other ACE inhibitors, angiotensin receptor blockers and beta-blockers”. The label for Diovan HCT states that the “the overall response to the combination was similar for black and non-black patients”. The label for Tekturna HCT noted that there were “too few non-Caucasians to assess differences in blood pressure effects by race.”

There is a need in the art for new methods of lowering blood pressure in black patients in which the blood pressure effect of drugs that are administered to black patients is similar to that in non-black patients.

### SUMMARY OF THE PRESENT INVENTION

In one embodiment, the present invention relates to a method of lowering blood pressure in a black patient in need of treatment thereof. The method comprises the steps of:

preferentially selecting a therapeutically effective amount of at least one angiotensin II receptor blocker (ARB) from a class of ARBs to lower the blood pressure in a black patient, wherein the ARB selected is azilsartan medoxomil; and

administering a combination of a therapeutically effective amount of the azilsartan medoxomil and a therapeutically effective amount of chlorthalidone to lower the blood pressure of the black patient.

In one aspect, the patient in the above method is administered 20 mg once per day of azilsartan medoxomil. In another aspect, the patient in the above method is administered 40 mg once per day of azilsartan medoxomil. In still yet another aspect, the patient in the above method is administered 80 mg once per day of azilsartan medoxomil.

In one aspect, the chlorthalidone in the above method is administered simultaneously or sequentially with the azilsartan medoxomil. Alternatively, in the above method, the chlorthalidone and azilsartan medoxomil are administered as separate dosage forms. Still further alternatively, the chlorthalidone and azilsartan medoxomil used in the above method are administered in a single dosage form.

In the above method, the patient is administered 12.5 mg once per day of chlorthalidone. Alternatively, in the above method, the patient is administered 25 mg once per day of chlorthalidone.

In the above method, the patient is administered 20 mg of azilsartan medoxomil and 25.0 mg of chlorthalidone once per day. Alternatively, in the above method, the patient is administered 40 mg of azilsartan medoxomil and 25.0 mg of chlorthalidone once per day. Still further alternatively, in the above method, the patient is administered 80 mg of azilsartan medoxomil and 25.0 mg of chlorthalidone once per day. In the above method, the patient is administered 20 mg of azilsartan medoxomil and 12.5 mg of chlorthalidone once per day. Alternatively, in the above method, the patient is administered 40 mg of azilsartan medoxomil and 12.5 mg of chlorthalidone once per day. Still further alternatively, in the above method, the patient is administered 80 mg of azilsartan medoxomil and 12.5 mg of chlorthalidone once per day.

In the above method, the blood pressure being measured is systolic blood pressure. In the above method, the blood pressure being measured is diastolic blood pressure. In the above method, the blood pressure being measured is mean arterial blood pressure. In the above method, the blood pressure being measured is systolic blood pressure, diastolic blood pressure, mean arterial blood pressure or any combinations thereof.

In the above method, the patient suffers from hypertension, heart failure, stroke, chronic kidney disease, arrhythmia, peripheral artery disease, coronary artery disease (including, for example, myocardial infarction, angina, etc) or combinations thereof.

In the above method, the hypertension is stage 1 primary systolic hypertension. In the above method, the hypertension is stage 2 primary systolic hypertension.

In another embodiment, the present invention relates to a method of lowering blood pressure in a black patient in need of treatment thereof. The method comprises the steps of:

preferentially administering a combination of a therapeutically effective amount of at least one angiotensin II receptor blocker (ARB) from a class of ARBs and a therapeutically effective amount of chlorthalidone to lower the blood pressure in a black patient, wherein the ARB administered to the black patient is azilsartan medoxomil.

In one aspect, the patient in the above method is administered 20 mg once per day of azilsartan medoxomil. In another aspect, the patient in the above method is administered 40 mg once per day of azilsartan medoxomil. In still yet another aspect, the patient in the above method is administered 80 mg once per day of azilsartan medoxomil.

In one aspect, the chlorthalidone in the above method is administered simultaneously or sequentially with the azilsartan medoxomil. Alternatively, in the above method, the chlorthalidone and azilsartan medoxomil are administered as separate dosage forms. Still further alternatively, the chlorthalidone and azilsartan medoxomil used in the above method are administered in a single dosage form.

In the above method, the patient is administered 12.5 mg once per day of chlorthalidone. Alternatively, in the above method, the patient is administered 25 mg once per day of chlorthalidone.

In the above method, the patient is administered 20 mg of azilsartan medoxomil and 25.0 mg of chlorthalidone once per day. Alternatively, in the above method, the patient is administered 40 mg of azilsartan medoxomil and 25.0 mg of chlorthalidone once per day. Still further alternatively, in the above method, the patient is administered 80 mg of azilsartan medoxomil and 25.0 mg of chlorthalidone once per day. In the above method, the patient is administered 20 mg of azilsartan medoxomil and 12.5 mg of chlorthalidone once per day. Alternatively, in the above method, the patient is administered 40 mg of azilsartan medoxomil and 12.5 mg of chlorthalidone once per day. Still further alternatively, in the above method, the patient is administered 80 mg of azilsartan medoxomil and 12.5 mg of chlorthalidone once per day.

In the above method, the blood pressure being measured is systolic blood pressure. In the above method, the blood pressure being measured is diastolic blood pressure. In the above method, the blood pressure being measured is mean arterial blood pressure. In the above method, the blood pressure being measured is systolic blood pressure, diastolic blood pressure, mean arterial blood pressure or any combinations thereof.

In the above method, the patient suffers from hypertension, heart failure, stroke, chronic kidney disease, arrhythmia, peripheral artery disease, coronary artery disease (including, for example, myocardial infarction, angina, etc) or combinations thereof.

In the above method, the hypertension is stage 1 primary hypertension. In the above method, the hypertension is stage 2 primary hypertension.

### BRIEF DESCRIPTION OF THE DRAWINGS

FIG. 1 shows subgroup analyses of clinical systolic blood pressure (SBP) by baseline characteristics pursuant to the study described in Example 1. In FIG. 1, AZL-M/CLD=azilsartan-medoxomil/chlorthalidone, OLM/HCTZ=olmesartan/hydrochlorothiazide. Open circles (o) are treatment differences between AZL-M/CLD 40/25 mg and OLM/HCTZ. Closed circles () are the treatment differences between AZL-M/CLD 80/25 mg group and OLM/HCTZ. The median clinic SBP at baseline was 163.3 mm Hg. Baseline eGFR categories expressed as ml/min/1.73 m². *P<0.05 vs. OLM/HCTZ.

### DETAILED DESCRIPTION OF THE INVENTION

### I. Definitions

Section headings as used in this section and the entire invention herein are not intended to be limiting.

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used herein, the term “angiotensin II receptor blocker (ARB)” refers to those active agents that bind to the AT₁-receptor subtype of angiotensin II receptor but do not result in activation of the receptor. As a consequence of the blockade of the AT₁ receptor, these antagonists can be employed as antihypertensive agents. Examples of angiotensin II receptor blockers include AT₁ receptor antagonists having differing structural features, preferred are those with the non-peptidic structures. Examples of such compounds include valsartan (EP Patent No. 443983), losartan (EP Patent No. 253310), candesartan (EP Patent No. 459136), eprosartan (EP Patent No. 403159), irbesartan (EP Patent No. 454511), olmesartan (EP Patent No. 503785), tasosartan (EP Patent No. 539086), telmisartan (EP Patent No. 522314), azilsartan medoxomil (U.S. Pat. No. 7,157,584), etc.

As used herein, the term “about” is used synonymously with the term “approximately.” Illustratively, the use of the term “about” indicates that values slightly outside the cited values, namely, plus or minus 10%. Such dosages are thus encompassed by the scope of the claims reciting the terms “about” and “approximately.”

As used herein, the term “azilsartan medoxomil” refers to the ARB known as (5-Methyl-2-oxo-1,3-dioxol-4-yl)methyl 2-ethoxy-1-{[2′-(5-oxo-4,5-dihydro-1,2,4-oxadiazol-3-yl)biphenyl-4-yl]methyl}-1H-benzimidazole-7-carboxylate monopotassium salt or metabolites thereof (azilsartan medoxomil is a prodrug) which has the below formula I and is indicated for the treatment of hypertension to lower blood pressure. Azilsartan medoxomil is commercially available in 40 mg or 80 mg tablets. Azilsartan medoxomil and methods for making azilsartan medoxomil are described in U.S. Pat. No. 7,157,584, the contents of which are herein incorporated by reference.

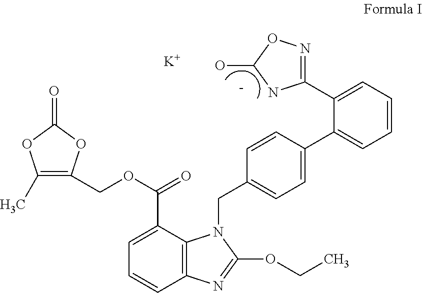 [SMILES: CCOc1nc2cccc(C(=O)OCc3oc(=O)oc3C)c2n1Cc1ccc(-c2ccccc2C2=NOC(O)N2)cc1 ; [K+]]

As used herein, the term “active agent” refers to an azilsartan medoxomil, chlorthalidone or combinations thereof.

The terms “administer”, “administering”, “administered” or “administration” refer to any manner of providing an active agent or drug (such as, azilsartan medoxomil, chlorthalidone or combinations thereof) to a subject or patient. Routes of administration can be accomplished through any means known by those skilled in the art. Such means include, but are not limited to, oral, buccal, intravenous, subcutaneous, intramuscular, transdermal, by inhalation and the like.

As used herein, the term “black” when used in connection with a subject refers to a subject having African ancestral origins. Such phrase is meant to include a subject having ancestral origins from multiple locations, not just Africa. For example, this term is meant to include a subject having ancestral origins from both the United States and from Africa, a subject having ancestral origins from a member country of the European Union and from Africa, a subject having ancestral origins from the Middle East and from Africa, a subject having ancestral origins from India and from Africa, a subject having ancestral origins from China or any other part of Asia, and from Africa, a subject having ancestral origins from Japan and from Africa, a subject having ancestral origins from Latin America or South America and from Africa, a subject having ancestral origins from Australia and from Africa, a subject having ancestral origins from the Caribbean and from Africa, a subject having ancestral origins from Canada and from Africa, a subject having ancestral origins from Mexico and from Africa, a subject having ancestral origins from Russia and from Africa, etc. Also, the term “black” encompasses any individual who self-identifies his or herself as being of the “black” race.

As used herein the term “chlorthalidone” refers to 2-chloro-5-(2,3-dihydro-1-hydroxy-3-oxo-1H-isoindol-1-yl)benzene-sulfonamide having the below Formula II:

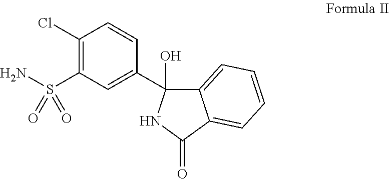 [SMILES: NS(=O)(=O)c1cc(C2(O)NC(=O)c3ccccc32)ccc1Cl]

Chlorthalidone inhibits the electroneutral Na⁺/Cl⁻ symporter in the distal convoluted tubule of the loop of Henle in the nephrons of the kidney, interfering with sodium reabsorption and leading to increased water excretion. Chlorthalidone tablets are commercially available containing either 25 mg or 50 mg of Chlorthalidone USP and the following inactive ingredients: colloidal silicon dioxide, microcrystalline cellulose, D&C Yellow #10, sodium starch glycolate, pregelatinized starch, stearic acid and other inactive ingredients. The 50 mg tablet also contains FD&C Blue #1.

The term “dosage form” refers to any solid object, semi-solid, or liquid composition designed to contain a specific pre-determined amount (i.e., dose) of a certain active agent. Suitable dosage forms may be pharmaceutical drug delivery systems, including those for oral administration, buccal administration, rectal administration, topical or mucosal delivery or subcutaneous implants, or other implanted drug delivery systems and the like. Preferably, the dosage forms described herein may be considered to be solid, however, they may contain liquid or semi-solid components. More preferably, the dosage form is an orally administered system for delivering an active agent to the gastrointestinal tract of a subject. The dosage form of the present invention may exhibit modified release of the active agent.

By an “effective amount” or a “therapeutically effective amount” of an active agent is meant a nontoxic but sufficient amount of the active agent to provide the desired effect. The amount of active agent that is “effective” will vary from subject to subject, depending on the age and general condition of the individual, the particular active agent or agents, and the like. Thus, it is not always possible to specify an exact “effective amount.” However, an appropriate “effective amount” in any individual case may be determined by one of ordinary skill in the art using routine experimentation.

As used herein, the phrase “diastolic blood pressure” refers to the minimum pressure exerted on the vessel walls when the heart muscle relaxes between beats and is filling with blood. Diastolic blood pressure is usually the second or bottom number in a blood pressure reading. Methods for measuring diastolic blood pressure are well known to those skilled in the art.

As used herein, the term or phrase “hypertension” or “elevated blood pressure” refers to a systolic blood pressure in a subject of at least 140 mmHg, a diastolic blood pressure in a subject of at least 90 mmHg, a mean arterial pressure of at least 106 mmHg or a combination of a systolic blood pressure of at least 140 mmHg and a diastolic blood pressure of at least 90 mmHg in a subject.

As used herein, the phrase “mean arterial blood pressure” “mean arterial pressure” or “MAP” refer to the product of cardiac output and peripheral vascular resistance. MAP is used to assess the hemodynamic status of a patient. More specifically, it is considered the perfusion pressure seen by organs in the body. Formulas for approximating MAP are well known to those skilled in the art. An example of a formula that can be used to calculate MAP is: MAP=2/3 diastolic blood pressure+1/3 systolic blood pressure

As used herein, the term “pre-hypertension” or “pre-hypertension blood pressure” refers to a systolic blood pressure in a subject in the range of 120 mmHg to 139 mmHg, a diastolic blood pressure in a subject in the range of 80 mmHg to 89 mmHg or a combination a systolic blood pressure in a subject in the range of 120 mmHg to 139 mmHg and a diastolic blood pressure in a subject in the range of 80 mmHg to 89 mmHg.

By “pharmaceutically acceptable,” such as in the recitation of a “pharmaceutically acceptable excipient,” or a “pharmaceutically acceptable additive,” is meant a material that is not biologically or otherwise undesirable, i.e., the material may be incorporated into a pharmaceutical composition administered to a patient without causing any undesirable biological effects.

The term “stage 1 primary systolic hypertension” refers to a systolic blood pressure in a subject of between 140-159 mmHg, inclusive.

The term “stage 2 primary systolic hypertension” refers to a systolic blood pressure in a subject of greater than or equal to 160 mmHg.

The term “subject” refers to an animal, preferably a mammal, including a human or non-human. The terms patient and subject may be used interchangeably herein.

As used herein, the term “systolic blood pressure” refers to the peak pressure exerted on the walls of the arteries during the contraction phase of the ventricles of heart. Systolic blood pressure is usually the first or top number in a blood pressure reading. Methods for measuring systolic blood pressure are well known to those skilled in the art.

The terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease. The phrase “preferentially treating a patient” or “preferentially treating the patient” as used herein refers to the reduction in severity and/or frequency of symptoms, elimination of symptoms and/or underlying cause, prevention of the occurrence of symptoms and/or their underlying cause, and improvement or remediation of damage that is achieved by administering to the patient or subject one or more specific active agents or drugs of a certain class over one or more active agents or drugs that are members of the same class. For example, in the context of the present invention, a black subject or black patient who is being treated for hypertension is preferentially treated with or administered azilsartan medoxomil instead of or in lieu of being treated with or administered one or more other ARBs such as, valsartan, losartan, candesartan, eprosartan, irbesartan, olmesartan, tasosartan, telmisartan (which are all ARBs). In other words, in the context of the present invention, patients or subjects are preferably administered azilsartan medoxomil in lieu of another ARB, such as, valsartan, losartan, candesartan, eprosartan, irbesartan, olmesartan, tasosartan, telmisartan etc. or any combinations of valsartan, losartan, candesartan, eprosartan, irbesartan, olmesartan, tasosartan, telmisartan, etc. Additionally, in the methods described herein, the azilsartan medoxomil is preferentially selected out of a class of ARBs which includes valsartan, losartan, candesartan, eprosartan, irbesartan, olmesartan, tasosartan, telmisartan or azilsartan medoxomil, etc for use in treating black patients suffering from hypertension as in the methods described herein. The preferentially selected azilsartan medoxomil is then used to treat a black patient in need of treatment thereof.

In addition, the preferential treatment of black patients with the azilsartan medoxomil according to the methods of the present invention is done in combination with treatment with chlorthalidone. The treatment with chlorthalidone is done simultaneously or sequentially with treatment with azilsartan medoxomil.

### II. The Present Invention

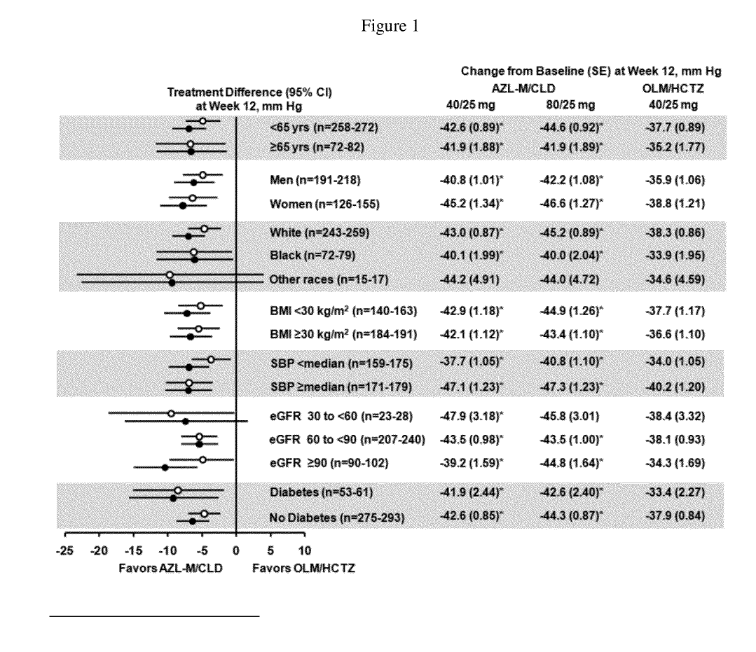

The present invention is based on the discovery that the administration of a therapeutically effective amount of azilsartan medoxomil in combination with a therapeutically effective amount of chlorthalidone to a black patient in need of treatment thereof results in a blood pressure lowering effect (namely, a reduction in one of more of systolic blood pressure, diastolic blood pressure, mean arterial blood pressure or combinations thereof) in said black patients that is similar (namely, is not statistically significantly different) to that of white patients (who have been administered the same combination of azilsartan medoxomil and chlorthalidone) (See, FIG. 1 and Example 1). In fact, administration of a therapeutically effective amount of azilsartan medoxomil in combination with a therapeutically effective amount of chlorthalidone has been found to be effective reducing blood pressure regardless of age, gender, or race.

In one embodiment, the present invention relates to methods of lowering blood pressure in black patients in need of treatment thereof. In one aspect, the method involves preferentially selecting a therapeutically effective amount of at least one ARB from a class of ARBs to lower blood pressure in a black patient. The class of ARBs from which at least one therapeutically effective ARB is selected from can, for example, comprise one or more of valsartan, losartan, candesartan, eprosartan, irbesartan, olmesartan, tasosartan, telmisartan, azilsartan medoxomil, etc. The number of ARBs and the types of ARBs that make of the class is not limited. Any therapeutic entity (small molecule, biologic, etc) that falls within the definition of an ARB provided herein can be included in the class of ARBs from which said preferential selection is made. From this class of ARBs, azilsartan medoxomil is preferentially selected over all the other ARBs in the class for use in the methods of the present invention.

Once the azilsartan medoxomil is preferentially selected from the other ARBs in the class, a therapeutically effective amount of the azilsartan medoxomil and a therapeutically effective amount of chlorthalidone are administered to the black patient to lower the blood pressure of the patient. In other words, in the methods of the present invention, the azilsartan medoxomil is preferentially administered over other ARBs to a black patient. This administration is performed in combination with a therapeutically effective amount of chlorthalidone.

The black patients being treated according to the method of the present invention may be suffering from one or number of diseases or medical conditions. For example, said black patients may be suffering from one or more of hypertension (such as stage 1 primary hypertension or stage 2 primary hypertension), heart failure, stroke, chronic kidney disease, arrhythmia, peripheral artery disease, coronary artery disease (myocardial infarction or angina) or any combinations thereof.

Methods for determining the blood pressure of a subject, such as a black patient, are well known in the art. For example, the systolic blood pressure and/or diastolic blood pressure of a subject can be determined using a sphygmomanometer (in mm of Hg) by a medical professional, such as a nurse or physician. Aneroid or electronic devices can also be used to determine the blood pressure of a subject and these devices and their use are also well known to those skilled in the art. Additionally, a 24-hour ambulatory blood pressure monitoring (hereinafter “ABPM”) device can be used to measure systolic blood pressure, diastolic blood pressure and heart rate. ABPM assesses systolic blood pressure, diastolic blood pressure and heart rate in predefined intervals (normally, the intervals are established at every 15 or 20 minutes, but any interval can be programmed) over a 24-hour period. The following parameters are then calculated from these readings after the data has been uploaded to a database. For example, ABPM can be used to measure the following: (1) the mean 24-hour systolic blood pressure of a subject; (2) the mean 24-hour diastolic blood pressure of a subject; (3) the mean daytime (The time period that constitutes “daytime” can readily be determined by those skilled in the art. For example, the “daytime” can be the time period from 6:00 a.m. until twelve noon or 7:00 a.m. to 10 p.m.) systolic blood pressure of a subject; (4) the mean daytime diastolic blood pressure of a subject; (4) the mean nighttime ((The time period that constitutes “nighttime” can readily be determined by those skilled in the art. For example, the “nightime” can be the time period from twelve midnight until 6:00 a.m. or 10:00 p.m. until 7:00 a.m.) systolic blood pressure of a subject; (5) the mean nighttime diastolic blood pressure of a subject; (6) the mean trough (The term “trough” refers to the time period at the end of the dosing period or the lowest point in drug levels and can readily be determined by those skilled in the art) systolic blood pressure of a subject; (7) the mean trough diastolic blood pressure of a subject; (8) the rate-pressure product (which is the product of heart rate and systolic blood pressure); and (9) the mean 24-hour mean rate-pressure product of a subject. The mean arterial pressure of a subject can be determined using a simple mathematical formula, such as the formula described previously herein (although alternative formulas are also known to those skilled in the art) once the systolic blood pressure and diastolic blood pressure of the subject has been determined. The time at which the blood pressure of the subject is determined is not critical for establishing the initial or baseline blood pressure reading. Once the initial or baseline blood pressure reading has been determined, a further determination is made by those skilled in the art as to whether or not the subject is suffering from (a) pre-hypertension or pre-hypertension blood pressure; or (b) hypertension or elevated blood pressure. For example, a baseline ABPM can be established 24-hours prior to beginning treatment of a subject in order to establish the initial or baseline ABPM in said subject. This initial or baseline APBM can also be used to determine whether or not the subject is suffering from pre-hypertension or hypertension. Additionally, central (aortic and carotid) blood pressure and central indices (including augmentation index, pulse pressure, and pulse wave velocity) can be measured using invasive and non-invasive devices. Central hemodynamic variables have been shown to be independently associated with organ damage, incident cardiovascular disease, and cardiovascular events in the general population and in specific disease states.

Any dosage of azilsartan medoxomil which, together with the chlorthalidone, provides a beneficial effect without unacceptable adverse side-effects in a black subject can be present in the combination, dosage form or composition, or used according to a methods of the present invention. While in one aspect the azilsartan medoxomil is administered orally, the invention is not limited to any route of administration, so long as the route selected results in effective delivery of the drug to provide a beneficial effect. Thus administration of the azilsartan medoxomil can illustratively be parenteral (e.g., intravenous, intraperitoneal, subcutaneous or intradermal), transdermal, transmucosal (e.g., buccal, sublingual or intranasal), intraocular, or rectal. Most conveniently for the majority of patients, however, the azilsartan medoxomil is administered orally, i.e., per os (p.o.). Any suitable orally deliverable dosage form can be used for the azilsartan medoxomil, including without limitation tablets, capsules (solid- or liquid-filled), powders, granules, syrups and other liquids, etc.

For oral administration, any dose of azilsartan medoxomil that, together with the chlorthalidone, is therapeutically effective, up to a maximum that is tolerated by the patient without unacceptable adverse side effects, can be administered. For most patients, such a dose is likely to be about 10 mg/day to about 200 mg/day, for example about 20 mg/day to about 100 mg/day or about 40 to about 80 mg/day. One of skill in the art can readily identify a suitable dose for azilsartan medoxomil from publicly available information in printed or electronic form, for example on the internet.

For oral administration, any dose of chlorthalidone, that, together with the azilsartan medoxomil, is therapeutically effective, up to a maximum that is tolerated by the patient without unacceptable adverse side effects, can be administered. For most patients, such a dose is likely to be about a suitable dose is likely to be about 2.5 to about 200 mg/day, for example about 6.25 mg/day to about 50 mg/day or about 12.5 mg/day to about 25 mg/day. One of skill in the art can readily identify a suitable dose for chlorthalidone from publicly available information in printed or electronic form, for example on the internet.

By way of example, for oral administration, a black patient can be administered 40 mg of azilsartan medoxomil and 12.5 mg of chlorthalidone. Alternatively, a black patient can be administered 80 mg of azilsartan medoxomil and 12.5 mg of chlorthalidone. Alternatively, a black patient can be administered 40 mg of azilsartan medoxomil and 25.0 mg of chlorthalidone. Alternatively, a black patient can be administered 80 mg of azilsartan medoxomil and 25.0 mg of chlorthalidone.

The prescribed daily dosage amount of the azilsartan medoxomil and chlorthalidone can be administered in any suitable number of individual doses, for example four times, three times, twice or once a day. With a dosage form having appropriate controlled release properties, a lower frequency of administration may be possible, for example once every two days, once a week, etc. Administration can be continued for as long as clinically necessary, or for any desired duration, for example as prescribed by a physician. Thus duration of administration can illustratively be about one week to about one year or longer, and in some situations can be continued for substantially the remaining duration of the life of the subject.

Azilsartan medoxomil is suitable for once a day administration, and, where chlorthalidone is likewise suitable for once a day administration, it is generally most convenient to administer both the azilsartan medoxomil and chlorthalidone once a day at around the same time, for example, orally in the dosage amounts desired. In other words, the azilsartan medoxomil and chlorthalidone can be administered once a day simultaneously (such as in a single (or fixed) dosage form) or sequentially (where the azilsartan medoxomil is part of a first dosage form and the chlorthalidone is part of a second dosage form and each of the first and second dosage forms is administered separately to a black patient with a period of time (which may be seconds or minutes). The order in which the first and second dosage forms are administered to the black patient is not critical. Further, the azilsartan medoxomil and chlorthalidone may be administered by the same or different routes of administration, and at the same or different times such as those listed above.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in separate containers, azilsartan medoxomil and chlorthalidone. In another example, the azilsartan medoxomil and chlorthalidone are separately packaged and available for sale independently of one another, but are co-marketed or co-promoted for use according to the invention. The separate dosage forms can also be presented to a subject separately and independently, for use according to the invention.

In another aspect of the invention, the combination can take the form of a pharmaceutical composition (dosage form) comprising the combination together with one or more pharmaceutically acceptable excipients. The composition can take any suitable form for the desired route of administration. Where the composition is administered orally, any suitable orally deliverable dosage form can be used, including without limitation tablets, capsules (solid- or liquid-filled), powders, granules, syrups and other liquids, etc.

Illustratively, a composition that is solid and orally deliverable, for example in a form of a tablet or capsule, typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function. Further information on excipients can be found in standard reference works such as Handbook of Pharmaceutical Excipients, 3rd ed. (Kibbe, ed. (2000), Washington: American Pharmaceutical Association).

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of .alpha.- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC), hydroxypropylcellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopalmitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used in compositions of the present invention. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

By way of example, and not of limitation, an example of the present invention shall now be given.

### Example 1

### Comparison of the Antihypertensive Effects of Azilsartan Medoxomil Plus Chlorthalidone Versus Olmesartan Medoxomil and Hydrochlorothiazide

### Primary Purpose:

The primary objective of this study was to compare the antihypertensive effect of azilsartan medoxomil potassium plus chlorthalidone (TAK-491CLD) FDC versus olmesartan medoxomil/hydrochlorothiazide (OLM/HCTZ) in subjects with moderate to severe hypertension.

### Secondary Purpose:

The secondary objectives were to evaluate the safety and tolerability of the TAK-491CLD fixed dose combination (FDC) versus OLM/HCTZ.

### Methodology

This was a phase 3b, double-blind, randomized, parallel-group, 12-week efficacy and safety study of the TAK-491CLD FDC versus OLM/HCTZ FDC in subjects with moderate to severe hypertension, defined as having a systolic blood pressure (SBP) between 160 and 190 mm Hg, inclusive. Randomization was stratified by subject's race: Black and non-Black. Before initiation of treatment, all subjects participated in a 2-week, single-blind, placebo Run-in Period (Days −14 to −1). Subjects who had not received antihypertensive treatment within 28 days before Screening were entered into the Run-in Period as soon as all inclusion and exclusion criteria, including laboratory results, were verified. Subjects taking previous antihypertensive agents were required to participate in a 3 week Washout Period (Days −21 to −1).

If the subject's previous antihypertensive treatment included amlodipine or chlorthalidone, then the washout was extended to 4 weeks (Days −28 to −1). The Screening Visit was scheduled before the Washout Run-in Period began so that laboratory test results were reviewed and subject eligibility was confirmed before other treatments were stopped or placebo was initiated.

Eligible subjects were randomly assigned to 12 weeks of treatment with 1 of the combination titration treatments listed in the following table. Each subject's dose was force-titrated at the end of Weeks 4 and 8.

| | | Second Dose- | |
|---|---|---|---|
| Treatment | First Dose-Starting | Starting at End of | Third Dose-Starting |
| Nomenclature | at Baseline | Week 4 | at End of Week 8 |
| TAK-491CLD | TAK-491CLD | Force-titrated to TAK- | Force-titrated to TAK- |
| 40/25 mg titration | 20/12.5 mg until end | 491CLD 40/12.5 mg | 491CLD 40/25 mg for |
| group (Treatment | of Week 4 | until end of Week 8 | the remainder of the |
| A) | | | study |
| TAK-491CLD | TAK-491CLD | Force-titrated to TAK- | Force-titrated to TAK- |
| 80/25 mg titration | 40/12.5 mg until end | 491CLD 80/12.5 mg | 491CLD 80/25 mg for |
| group (Treatment | of Week 4 | until end of Week 8 | the remainder of the |
| B) | | | study |
| OLM/HCTZ 40/25 | OLM/HCTZ 20/12.5 | Force-titrated to | Force-titrated to |
| mg titration group | mg until end of Week | OLM/HCTZ 40/12.5 | OLM/HCTZ 40/25 mg |
| (Treatment C) | 4 | mg until end of Week 8 | for the remainder of the |
| | | | study |

Throughout the study, each treatment group received a dose of 3 tablets and 1 capsule, all given once dally (QD); TAK-491CLD (or matching placebo) was administered in tablet form, whereas OLM/HCTZ (or matching placebo) was administered as an individual (overencapsulated) capsule.

Sitting trough clinic blood pressure readings (hereinafter simply referred to as clinic blood pressure), standing blood pressure readings, and pulse measurements were measured in the clinic throughout the study, while 24-hour ambulatory blood pressure monitoring (ABPM) was conducted twice. Subjects were required to have a baseline (Day −1) ABPM reading that met predefined quality control criteria in order to be eligible for randomization. The final ABPM began at Week 12 (Visit 10) (ie, after administration of the last dose of study drug).

### Number of Subjects:

Planned: 1050 subjects (350 per arm).

### Analyzed:

Full Analysis Set (FAS) and Safety Analysis Set (SAS)-1071 subjects: 355 subjects in the TAK-491CLD 40/25 mg titration group, 352 subjects in the TAK-491CLD 80/25 mg titration group, and 364 subjects in the OLM/HCTZ titration group.

Per Protocol Analysis Set (PPS)-952 subjects: 315 subjects in the T AK-491 CLD 40/25 mg titration group, 313 subjects in the TAK-491CLD 80/25 mg titration group, and 324 subjects in the OLM/HCTZ titration group.

### Diagnosis and Main Criteria for Inclusion:

To qualify for study participation, subjects must have been diagnosed with moderate to severe essential hypertension, defined as a SBP between 160 and 190 mm Hg, inclusive; aged 18 years and older; been able to comprehend and willing to sign an informed consent form; been willing to discontinue current antihypertensive medications for up to 28 days prior to randomization; and had a clinic diastolic blood pressure (DBP)<119 mm Hg on Day −1.

### Test Product, Dose and Mode of Administration/Lot Number:

TAK-491CLD 20/12.5 mg, tablets, oral
TAK-491CLD 40/12.5 mg, tablets, oral
TAK-491CLD 40/25 mg, tablets, oral
TAK-491CLD 80/12.5 mg, tablets, oral
TAK-491CLD 80/25 mg, tablets, oral

### Duration of Treatment:

The study included a Washout/Single-Blind Placebo Run-In Period of up to 4 weeks depending on the previous antihypertensive agents taken by the subject, a 12-week Treatment Period during which subjects were force-titrated at the end of Weeks 4 and 8, and a 2-week Follow-Up Period that concluded with a phone call.

### Reference Therapy, Dose and Mode of Administration, Batch Number:

TAK-491CLD placebo, tablets, oral
OLM/HTCZ placebo, capsules, oral
OLM/HTCZ 20/12.5 mg, tablets, oral
OLM/HTCZ 40/12.5 mg, tablets, oral
OLM/HTCZ 40125 mg, tablets, oral
OLM/HTCZ 20/12.5 mg, overencapsulated tablets, oral
OLM/HTCZ 40/12.5 mg, overencapsulated tablets, oral
OLM/HTCZ 40125 mg, overencapsulated tablets, oral

### Criteria for Evaluation:

Primary efficacy endpoint: change from Baseline to Week 12 in mean clinic SBP. Secondary efficacy endpoints: change from Baseline to Weeks 4 and 8 in clinic SBP, change from Baseline to Weeks 4, 8, and 12 in clinic DBP, change from Baseline to Week 12 in mean systolic and diastolic ABPM parameters (trough [22- to 24-hours after dosing], 24-hour mean, daytime [6 AM-10 PM], nighttime [12 AM-6 AM], O— to 12-hour, and during each I-hour interval of the 24-hour ABPM), and the percentage of subjects at Weeks 4, 8, and 12 who reached their blood pressure target (as defined by clinic SBP <140 mm Hg or a reduction of >20 mm Hg from Baseline, or clinic DBP <90 mm Hg or a reduction of >10 mm Hg from Baseline, or both of the above).

### Safety:

Safety variables included adverse events, vital signs (including orthostatic vital signs), 12-lead electrocardiogram (ECG) findings, and clinical laboratory test results. (Note: All serum creatinine elevations that increased from Baseline ≧30% and >upper limit of normal [ULN] were to be reported as an adverse event. Investigators were advised to discontinue subjects with creatinine values persistently increased ≧50% from Baseline and >ULN.)

### Statistical Methods:

The FAS was the primary data set used for efficacy analyses. Efficacy analyses based on the PPS were also performed when appropriate. All routine safety analyses were based on the SAS. Analyses of the primary and secondary variables, excluding ABPM, were performed on last observation carried forward (LOCF) data set. In the LOCF analysis data set, the last postbaseline double-blind observed value was carried forward and used for all subsequent scheduled time points where data were missing (eg, the subject had missing data or dropped out of the study). Sensitivity analyses on trough clinic SBP and DBP were performed on observed values and using multiple imputation for missing trough clinic blood pressure data to assess the impact of LOCF methodology and drop-outs.

The primary analysis was based on an analysis of covariance (ANCOV A) model for change from Baseline to Final Visit for the primary efficacy variable. The model included treatment as a factor and Baseline clinic SBP as a covariate. Estimates of treatment differences along with P-value and 95% confidence interval (CI) were obtained from the ANCOV A model. The overall type 1 error rate of 0.05 was controlled using the principle of “closed” testing. Under this principle, the hypothesis of “all titration groups equal” was first tested at the 0.05 significance level. Upon rejection of this hypothesis, the 2 pairwise comparisons (Treatments A versus C and Treatment B versus C) were tested at the 0.05 level.

Change from Baseline in clinic SBP at Weeks 4 and 8 was analyzed using ANCOVA analysis and the “closed” testing procedure for controlling type I error described above. A similar analysis was used for all other secondary endpoints, except for the analysis of percentage of subjects responding to treatment, which used a logistic model with treatment as a fixed effect and baseline clinic blood pressure as a covariate to analyze the response criteria for that clinic value for each study visit. The odds ratio and its 95% CI using the logistic model estimates were estimated.

Treatment-emergent adverse events (TEAEs) were tabulated, and vital signs and 12-lead ECG findings were summarized with descriptive statistics. All safety data were presented in the listings.

Additional analyses were performed for subgroups based on age, sex, race, and other important baseline factors.

### Summary of Results

### Subject Disposition:

A total of 1071 subjects (mean age of 56.6 years), including 629 male and 442 female subjects, were randomized in the study from the 2933 subjects who were screened. Of the 1071 subjects, 892 (83.3%) subjects completed the study. A total of 179 (16.7%) subjects permanently discontinued the study (55 subjects [15.5%] and 77 subjects [21.9%] in the TAK-491CLD 40/25 and 80/25 mg titration groups, respectively, and 47 subjects [12.9%] in the OLM/HCTZ titration group). The most common reasons for premature withdrawal were adverse event (9.8%) and voluntary withdrawal (3.9%). The percentage of subjects who discontinued treatment due to an adverse event was similar in the TAK-491CLD 40/25 mg titration group (7.9%) and the OLM/HCTZ titration group (7.1%), but higher in the TAK-491CLD 80/25 mg titration group (14.5%). The numbers of subjects who enrolled and prematurely discontinued, along with primary reasons for premature discontinuation, are summarized in the following Table 1

TABLE 1

| | TAK-491-CLD | | OLM/HCTZ |
|---|---|---|---|
| | 20/12.5→ | 0/12.5→ | 20/12.5→ |
| | 40/12.5→ | 80/12.5→ | 40/12.5→ |
| | 40/25 mg | 80/25 mg | 40/25 mg |
| | N = 355 | N = 352 | N = 364 |
| Number of subjects randomized | 355 | 352 | 364 |
| Number (%) of subjects | 55 (15.5) | 77 (21.9) | 47 (12.9) |
| who prematurely | | | |
| discontinued study medication | | | |
| Primary reason for discontinuation | | | |
| Adverse events | 28 (7.9) | 51 (14.5) | 26 (7.1) |
| Protocol deviation | 0 | 2 (0.6) | 1 (0.3) |
| Lost to follow-up | 4 (1.1) | 5 (1.4) | 5 (1.4) |
| Voluntary withdrawal | 17 (4.8) | 14 (4.0) | 11 (3.0) |
| Lack of efficacy | 0 | 1 (0.3) | 1 (0.3) |
| Other | 6 (1.7) | 4 (1.1) | 3 (0.8%) |

### Baseline Demographics and Characteristics:

The demographic and baseline characteristics, summarized in the following Table 2, were relatively similar between treatment groups in all randomized subjects.

TABLE 2

| | TAK-491-CLD | | | | OLM/HCTZ | |
|---|---|---|---|---|---|---|
| | 20/12.5→ | | 0/12.5→ | | 20/12.5→ | |
| | 40/12.5→ | | 80/12.5→ | | 40/12.5→ | |
| | 40/25 mg | | 80/25 mg | | 40/25 mg | |
| Characteristic | N = 355 | | N = 352 | | N = 364 | |
| Sex, n (%) | | | | | | |
| Males | 223 | (62.8) | 201 | (57.1) | 205 | (56.3) |
| Females | 132 | (37.2) | 151 | (42.9) | 159 | (43.7) |
| Age, years | | | | | | |
| Mean (SD) | 56.4 | (10.49) | 56.7 | (10.07) | 56.7 | (10.86) |
| Age Categories, n (%) | | | | | | |
| <45 years | 46 | (13.0) | 33 | (9.4) | 46 | (12.6) |
| 45 to 64 years | 235 | (66.2) | 241 | (68.5) | 234 | (64.3) |
| ≧65 years | 74 | (20.8) | 78 | (22.2) | 84 | (23.1) |
| Region, n (%) | | | | | | |
| US | 276 | (77.7) | 277 | (78.7) | 285 | (78.3) |
| Canada | 79 9 | (22.3) | 75 | (21.3) | 79 | (21.7) |
| Ethnicity, n (%)(a) | | | | | | |
| Non-Hispanic or Latino | 248 | (69.9) | 241 | (68.5) | 251 | (69.0) |
| Hispanic or Latino | 28 | (7.9) | 36 | (10.2) | 34 | (9.3) |
| Missing | 79 | (22.3) | 75 | (21.3) | 79 | (21.7) |
| Race, n. (%)(b) | | | | | | |
| White | 261 | (73.5) | 258 | (73.3 | 267 | (73.4) |
| Black | 80 | (22.5) | 80 | (22.7) | 80 | (22.0) |
| Asian | 13 | (3.7) | 11 | (3.1) | 11 | (3.0) |
| American Indian or Alaska Native | 2 | (0.6) | 5 91.4) | | 5 | (1.4) |
| Native Hawaiian or Other Pacific | 0 | | 1 | (0.3) | 2 | (0.5) |
| Islander | | | | | | |
| Multiracial | 1 | (0.3) | 2 | (0.6) | 1 | (0.3) |
| Weight, kg | | | | | | |
| Mean (SD) | 92.34 | | 91.47 | | 90.83 | |
| | (20.296) | | (20.987) | | (18.590) | |
| Height, cm | | | | | | |
| Mean (SD) | 171.2 | | 169.3 | | 169.4 | |
| BMI, kg/m² | (10.15) | | (10.88) | | (10.15) | |
| Mean (SD) | 31.4 | (5.94) | 31.9 | (6.59) | 31.6 | (5.92) |
| Estimated Glomerular Filtration Rate | | | | | | |
| Category, mL/min/1.73 m², n (%) | | | | | | |
| Severe impairment: ≧0 to >30 | 0 | | 0 | | 1 | (0.3) |
| Moderate impairment ≧30 to 60 | 26 | (7.3) | 29 | (8.2) | 25 | (6.9) |
| Mild impairment ≧60 to <90 | 224 | (63.1) | 220 | (62.5) | 246 | (67.6) |
| Normal function ≧90 | 105 | (29.6) | 103 | (29.3) | 91 | (25.0) |

| | | | | |
|---|---|---|---|---|
| Missing | 0 | 0 | 1 | (0.3) |

(a)Ethnicity was only collected at United States sites.
(b)A subject could have chosen more than 1 category for race; if so, they were included in each category indicated and were also included in the multiracial category.

### Efficacy Results:

Statistically significantly greater changes from Baseline to Week 12 in clinic SBP were observed for both the TAK-491CLD 40/25 and 80/25 mg titration groups (−42.5 and −44.0 mm Hg, respectively) compared with the OLM/HCTZ titration group (−37.1 mm Hg); the treatment difference and corresponding 95% CI were −5.3 (−7.6, −3.1) mm Hg (P<0.001) in favor of the TAK-491CLD 40/25 mg titration group and −6.9 (−9.2, −4.6) mm Hg (P<0.001) in favor of the 80/25 mg titration group. The above pairwise comparisons were performed as the second step of a closed testing procedure, where the initial test for an overall treatment difference among 3 groups was first found to be statistically significant (P<0.001).

Within each titration group, clinically significant incremental reductions were observed, with the majority of effect seen by Week 2; statistically significantly greater reductions in both TAK-491CLD titration groups compared with the OLM/HCTZ titration group were observed at each postbaseline visit.

At Week 4, the actual doses were TAK-491CLD 20/12.5 mg, TAK-491CLD 40/12.5 mg, and OLM/HCTZ 20/12.5 mg. Within the closed testing procedure, statistically and clinically significantly greater changes from Baseline to Week 4 in clinic SBP were observed for the TAK-491CLD 40/25 and 80/25 mg titration groups (−34.7 and −36.7 mm Hg, respectively) than with the OLM/HCTZ titration group (−29.7 mm Hg); the treatment differences and corresponding 95% CIs were −5.0 (−7.1, −2.9) mm Hg (P<0.001) in favor of the TAK-491CLD 40/25 mg titration group and −7.0 (−9.2, −4.8) mm Hg (P<0.001) in favor of the 80/25 mg titration group. At Week 8, the actual doses were TAK-491CLD 40/12.5 mg, TAK-491CLD 80/12.5 mg, and OLM/HCTZ 40/12.5 mg. Within the closed testing procedure, statistically and clinically significantly greater changes from Baseline to Week 8 in clinic SBP were observed for the TAK-491CLD 40/25 and 80/25 mg titration groups (−39.1 and −39.4 mm Hg, respectively) than with the OLM/HCTZ titration group (−33.5 mm Hg); the treatment differences and corresponding 95% CIs were −5.6 (−7.8, −3.5) mm Hg (P<0.001) in favor of the TAK-491CLD 40/25 mg titration group and −5.9 (−8.0, −3.7) mm Hg (P<0.001) in favor of the 80/25 mg titration group. Therefore, both TAK-491CLD titration groups were more efficacious than the OLM/HCTZ titration group at Week 4 and at Week 8 within the framework of the closed testing scheme.

For clinic DBP, statistically and clinically significantly greater changes from Baseline to Week 12 were observed for both TAK-491CLD 40/25 and 80/25 mg titration groups compared with the OLM/HCTZ titration group. Results for the clinic SBP and DBP changes from Baseline are shown in the following table.

Results from sensitivity analyses using a multiple imputation method for missing data, as well as analyses using the PPS, were consistent with the analyses of the F AS described above for clinic measures of SBP and DBP.

TABLE 3

| | TAK-491-CLD | | OLM/HCTZ |
|---|---|---|---|
| | 20/12.5→ | 0/12.5→ | 20/12.5→ |
| | 40/12.5→ | 80/12.5→ | 40/12.5→ |
| | 40/25 mg | 80/25 mg | 40/25 mg |
| Study Visit | N = 355 | N = 352 | N = 364 |

Clinic SBP Results

| Baseline (a) | | | | | | |
|---|---|---|---|---|---|---|
| n | 344 | | 330 | | 354 | |
| LS mean (SE) | 164.8 | (0.54) | 165.0 | (0.56) | 164.6 | (0.54) |
| P-value vs OLM/HCTZ | 0.777 | | 9,593 | | | |
| Week 4 (Secondary Endpoint) | | | | | | |
| n | 343 | | 330 | | 352 | |
| LS mean Δ from BL (SE) | −34.7 | (0.77) | −36.7 | (0.79) | −29.7 | (0.76) |
| LS mean difference vs | −5.0 | | −7.0 | | | |
| OLM/HCTZ | | | | | | |
| (95% CI) | (−7.1, −2.9) | | (−9.2, −4.8) | | | |
| P-value | <0.000* | | <0.001* | | | |
| Week 8 (Secondary Endpoint) | | | | | | |
| n | 344 | | 330 | | 353 | |
| LS mean Δ from BL (SE) | −39.1 | (0.78) | −39.4 | (0.80) | −33.5 | (0.77) |
| LS mean difference vs | −5.6 | | −5.9 | | | |
| OLM/HCTZ | | | | | | |
| (95% CI) | (−7.8, −3.5) | | (−8.0, −3.7) | | | |
| P-value | <0.001* | | <0.001* | | | |
| Week 12 (Primary Endpoint) | | | | | | |
| n | 344 | | 330 | | 354 | |
| LS mean Δ from BL (SE) | −42.5 | (0.81) | −44.0 | (0.83) | −37.1 | (9.80) |

| | | |
|---|---|---|
| LS mean difference vs | −5.3 | −6.9 |
| OLM/HCTZ | | |
| (95% CI) | (−7.6, −3.1) | (−9.2, −4.6) |
| P-value | <0.001* | <0.001* |

Clinic DBP Results

| Baseline (a) | | | | | | |
|---|---|---|---|---|---|---|
| n | 344 | | 330 | | 354 | |
| LS mean (SE) | 96.1 | (0.54) | 95.9 | (0.55) | 95.3 | (0.53) |
| P-value vs OLM/HCTZ | 0.270 | | 0.398 | | | |
| Week 4 (Secondary Endpoint) | | | | | | |
| n | | | | | | |
| LS mean Δ from BL (SE) | −14.9 | (0.46) | −15.8 | (0.47) | −11.7 | (0.45) |
| LS mean difference vs. | −3.2 | | −4.1 | | | |
| OLM/HCTZ | | | | | | |
| (95% CI) | (−4.4, −1.9) | | (−5.4, −2.8) | | | |
| P-value | <0.001* | | <0.001* | | | |
| Week 8 (Secondary Endpoint) | | | | | | |
| n | 344 | | 330 | | 353 | |
| LS mean Δ from BL (SE) | −17.0 | (0.48) | −17.7 | (0.49) | −13.9 | (0.48) |
| LS mean difference vs. | −3.0 | | −3.8 | | | |
| OLM/HCTZ | | | | | | |
| (95% CI) | (−4.4, −1.7) | | (−5.1, −2.4) | | | |
| P-value | <0.001* | | <0.001* | | | |
| Week 12 (Secondary Endpoint) | | | | | | |
| n | 344 | | 330 | | 354 | |
| LS mean Δ from BL (SE) | −18.8 | (0.47) | −20.5 | (0.48) | −16.4 | (0.46) |

| | | |
|---|---|---|
| LS mean difference vs. | −2.3 | −4.1 |
| OLM/HCTZ | | |
| (95% CI) | (−3.6, −1.0) | (−5.4, −2.8) |
| P-value | <0.001* | <0.001* |

LS = least squares, Δ from BL = Change from Baseline.
Note:
Postbaseline P-values are obtained from an ANCOVA model with treatment as a factor and Baseline as a covariate. All results are given as LOCF.
*Indicates statistically significant difference versus OLM/HCTZ at the 0.05 level.
(a) Baseline value is the last observation before the first dose of double-blind study drug.

Statistically significantly greater decreases from Baseline were observed in both the TAK-491CLD 40/25 and 80/25 mg titration groups compared with the OLM/HCTZ titration group for each mean systolic and diastolic ABPM parameter at Week 12 (ie, change from Baseline in LS mean trough [22- to 24-hours after dosing], 24-hour mean, mean daytime [6 AM-10 PM], mean nighttime [12 AM-6 AM], and mean at 0- to 12-hours after dosing), and at each hour throughout the 24-hour interval. The results are shown below in Tables 4A and 4B:

TABLE 4A
Changes From Baseline in Systolic ABPM Parameters
at Week 12 (mm Hg) (LOCF, FAS)

| | TAK-491-CLD | | OLM/HCTZ |
|---|---|---|---|
| | 20/12.5→ | 40/11.5→ | 20/12.5→ |
| | 40/12.5→ | 80/12.5→ | 40/12.5→ |
| | 40/25 mg | 80/25 mg | 40/25 mg |
| ABPM by Study Visit | N = 355 | N = 352 | N = 364 |

[symbol]

| | | | |
|---|---|---|---|
| Baseline [symbol] | | | |
| N | 232 | 234 | 238 |
| LS mean (SE) | 154.4 [symbol] | 156.6 [symbol] | 154.3 (1.01) |
| P-value [symbol] | [symbol] | 0.112 | |
| Week 12 | | | |
| N | 232 | 234 | 238 |
| LS mean [symbol] from BL (SE) | [symbol] | [symbol] | [symbol] |
| LS mean difference vs [symbol] | −7.0 | −9.0 | |
| [symbol] | [symbol] | [symbol] | |
| p-value | <0.001* | <0.001* | |

24-hour Mean SBP

| Baseline [symbol] | | | | | |
|---|---|---|---|---|---|
| [symbol] | 232 | | 214 | | 238 |
| LS mean (SE) | 150.4 | (0.85) | 152.2 | (0.88) | [symbol] |

| | | | |
|---|---|---|---|
| p-value vs [symbol] | [symbol] | [symbol] | |
| Week 12 | | | |
| [symbol] | 232 | 214 | 238 |
| LS mean [symbol] | [symbol] | [symbol] | [symbol] |
| LS mean difference vs [symbol] | [symbol] | [symbol] | |
| [symbol] | [symbol] | [symbol] | |
| p-value | <0.001* | <0.001* | |

Daytime

[symbol]

| | | | |
|---|---|---|---|
| Baseline (a) | | | |
| n | 232 | 214 | 238 |
| LS mean [symbol] | [symbol] | [symbol] | [symbol] |
| p-value vs [symbol] | [symbol] | [symbol] | |
| Week 12 | | | |
| n | 232 | 214 | 238 |
| LS mean [symbol] from BL ( [symbol] ) | [symbol] | [symbol] | [symbol] |
| LS mean difference [symbol] | [symbol] | [symbol] | |
| [symbol] | [symbol] | [symbol] | |
| p-value | [symbol] | [symbol] | |

Nighttime SBP (12 AM-6 AM)

| | | | |
|---|---|---|---|
| Baseline (a) | | | |
| n | 232 | 214 | 238 |
| LS mean (SE) | [symbol] | [symbol] | [symbol] |
| [symbol] | [symbol] | [symbol] | |
| Week 12 | | | |
| n | 232 | 214 | 238 |
| LS mean [symbol] | [symbol] | [symbol] | [symbol] |
| LS Mean difference [symbol] | [symbol] | [symbol] | |
| [symbol] | [symbol] | [symbol] | |
| p-value | [symbol] | [symbol] | |

SBP at 8 to 12 hours

| | | | |
|---|---|---|---|
| Baseline (a) | | | |
| n | 232 | 214 | 238 |
| LS Mean [symbol] | [symbol] | [symbol] | [symbol] |
| p-value [symbol] | [symbol] | [symbol] | |
| Week 12 | | | |
| n | 232 | 234 | 238 |
| LS mean [symbol] | [symbol] | [symbol] | [symbol] |
| LS mean difference [symbol] | [symbol] | [symbol] | |
| [symbol] | [symbol] | [symbol] | |
| p-value | [symbol] | [symbol] | |

* [symbol]
Note:

[symbol]

(a) [symbol] [symbol]
indicates data missing or illegible when filed

TABLE 4B

| | TAK-491-CLD | | OLM/HCTZ |
|---|---|---|---|
| | 20/12.5→ | 0/12.5→ | 20/12.5→ |
| | 40/12.5→ | 80/12.5→ | 40/12.5→ |
| | 40/25 mg | 80/25 mg | 40/25 mg |
| Study Visit | N = 355 | N = 352 | N = 364 |

Diastolic ABPM Results
Trough DBP (22-24-hours)

| Baseline (a) | | | | | | |
|---|---|---|---|---|---|---|
| n | 232 | | 214 | | 238 | |
| LS mean (SE) | 93.1 | (0.78) | 93.3 | (0.81) | 92.2 | (0.77) |
| P-value vs OLM/HCTZ | 0.450 | | 0.351 | | | |
| Week 12 | | | | | | |
| n | 232 | | 214 | | 238 | |
| LS mean Δ from BL (SE) | −19.8 | (0.55) | −20.2 | (0.57) | −16.0 | (0.54) |

| | | |
|---|---|---|
| LS mean difference vs | −3.9 | −4.3 |
| OLM/HCTZ | | |
| (95% CI) | (−5.4, −2.4) | (−5.8, −2.7) |
| P-value | <0.001* | <0.001* |

24-hour Mean DBP

| Baseline (a) | | | | | | |
|---|---|---|---|---|---|---|
| n | 232 | | 214 | | 238 | |
| LS mean (SE) | 88.3 | (0.69) | 88.9 | (0.72) | 87.4 | (0.68) |
| P-value vs OLM/HCTZ | 0.308 | | 0.109 | | | |
| Week 12 | | | | | | |
| n | 232 | | 214 | | 238 | |
| LS mean Δ from BL (SE) | −19.4 | (0.45) | −20.7 | (0.46) | −16.2 | (0.44) |

| | | |
|---|---|---|
| LS mean difference vs | −3.2 | −4.5 |
| OLM/HCTZ | | |
| (95% CI) | (−4.5, −2.0) | (−5.8, −3.3) |
| P-value | <0.001* | <0.001* |

Daytime DBP (6 am-10 pm)

| Baseline (a) | | | | | | |
|---|---|---|---|---|---|---|
| n | 232 | | 214 | | 238 | |
| LS mean (SE) | 91.7 | (0.72) | 92.4 | (0.75) | 90.9 | (0.71) |
| P-value vs OLM/HCTZ | 0.417 | | 0.147 | | | |
| Week 12 | | | | | | |
| n | 232 | | 214 | | 238 | |
| LS mean Δ from BL (SE) | −20.1 | (0.48) | −21.8 | (0.50) | −17.0 | (0.47) |

| | | |
|---|---|---|
| LS mean difference vs | −3.1 | −4.8 |
| OLM/HCTZ | | |
| (95% CI) | (−4.4, −1.8) | (−6.1, −3.4) |
| P-value | <0.001* | <0.001* |

Nighttime DBP (12 am-6 am)

| Baseline (a) | | | | | | |
|---|---|---|---|---|---|---|
| n | 232 | | 214 | | 238 | |
| LS mean (SE) | 78.7 | (0.74) | 79.3 | (0.77) | 77.2 | (0.73) |
| P-value vs OLM/HCTZ | 0.132 | | 0.045* | | | |
| Week 12 | | | | | | |
| n | 232 | | 214 | | 238 | |
| LS mean Δ from BL (SE) | −17.5 | (0.48) | −18.0 | (0.50) | −14.0 | (0.48) |

| | | |
|---|---|---|
| LS mean difference vs | −3.5 | −4.0 |
| OLM/HCTZ | | |
| (95% CI) | (−4.8, −2.2) | (−5.4, −2.7) |
| P-value | <0.001* | <0.001* |

DBP at 0- to 12-hours

| Baseline (a) | | | | | | |
|---|---|---|---|---|---|---|
| N | 232 | | 214 | | 238 | |
| LS mean (SE) | 92.5 | (0.74) | 93.1 | (0.77) | 91.6 | (0.73) |
| P-value vs OLM/HCTZ | 0.383 | | 0.152 | | | |
| Week 12 | | | | | | |
| N | 232 | | 214 | | 238 | |
| LS mean Δ from BL (SE) | −20.4 | (0.51) | −22.2 | (0.53) | −17.5 | (0.50) |

| | | |
|---|---|---|
| LS mean difference vs | −2.9 | −4.8 |
| OLM/HCTZ | | |
| (95% CI) | (−4.3, −1.5) | (−6.2, −3.3) |
| P-value | <0.001* | <0.001* |

Note:
Postbaseline P-values are obtained from an ANCOVA model with treatment as a fixed effect and Baseline as a covariate.
*Indicates statistically significant difference versus OLM/HCTZ at the 0.05 level. All results are given as LOCF.
(a) Baseline value is the last observation before the first dose of double-blind study drug.

The percentage of subjects in the FAS who responded to treatment as determined by clinic SBP <140 mm Hg or a reduction of ≧20 mmHg from Baseline), clinic DBP <90 mm Hg or a reduction of ≧10 mm Hg from Baseline), and joint SBP and DBP criteria (SBP <140 mm Hg or a reduction from Baseline SBP of ≧20 mm Hg and DBP <90 mm Hg or a reduction from Baseline DBP of ≧10 mm Hg) were analyzed at Weeks 4, 8, and 12. For the SBP response criteria, the TAK-491CLD 80/25 mg titration group compared with the OLM/HCTZ titration group achieved a statistically significantly greater percentage of responders at all visits, while the TAK-491CLD 40/25 mg titration group compared with the OLM/HCTZ titration group achieved a statistically significantly greater percentage of responders at Weeks 4 and 8. At Week 12, 93.0% and 94.2% of subjects in the TAK-491CLD 40/25 and 80/25 mg titration groups, respectively, had responded compared with 89.3% in the OLM/HCTZ titration group. For the joint SBP and DBP response criteria, both TAK-491CLD titration groups compared with the OLM/HCTZ titration group achieved a statistically significantly greater percentage of responders at Weeks 4, 8, and 12. At Week 12, 91.3% and 92.4% of subjects in the TAK-491CLD 40/25 and 80/25 mg titration groups had responded compared with 84.7% in the OLM/HCTZ titration group. For the DBP response criteria, the T AK-491CLD 80/25 mg titration group was statistically significantly different compared with the OLM/HCTZ titration group at Week 12. At Week 12, 94.5% and 95.8% of subjects in the T AK-491 CLD 40/25 and 80/25 mg titration groups, respectively, had responded compared with 91.5% in the OLM/HCTZ titration group. Similar analyses of the percentage of subjects who achieved target blood pressure at each visit were consistent with the responder analyses.

### Subgroup Analysis

Subgroup analyses were conducted by age, sex, race, as well as baseline BMI, eGFR, diabetes status, baseline SBP (above or below study median), and hypertension severity (both SBP and DBP) to evaluate for heterogeneity of blood pressure effects within and between titration groups. Statistical analyses were performed comparing TAK-491CLD with OLM/HCTZ within a subgroup level and the results are shown in the below Table 5 (also, see FIG. 1).

TABLE 5
Subgroup Analysis of Change From Baseline to Week 12 in Clinic SBP
(LOCF, FAS)

| | TAK-491 CLD | | OLM/HCTZ |
|---|---|---|---|
| | 20/12.5 mg → | 40/12.5 mg → | 20/12.5 mg → |
| | 40/12.5 mg → | 80/12.5 mg → | 40/12.5 mg → |
| Subgroup | 40/25 mg → | 80/25 mg → | 40/25 mg → |

Age

| <65 years | | | | | | |
|---|---|---|---|---|---|---|
| n | 271 | | 258 | | 272 | |
| LS mean at Baseline (SE) (a) | 164.2 | (0.59) | 164.7 | (0.60) | 164.5 | (0.59) |
| LS mean Δ from BL (SE) | −42.6 | (0.89) | −44.6 | (0.92) | −37.7 | (0.89) |
| LS mean difference vs OLM/HCTZ | −4.9 | | −6.9 | | | |
| (95% CI) | (−7.4, −2.4) | | (−9.4, −4.4) | | | |
| P-value | <0.001ⁱ | | <0.001* | | | |
| ≧65 years | | | | | | |
| n | 73 | | 72 | | 82 | |
| LS mean at Baseline (SE) (a) | 167.1 | (1.34) | 166.3 | (1.35) | 165.1 | (1.27) |
| LS mean Δ from BL (SE) | −41.9 | (1.88) | −41.9 | (1.89) | −35.2 | (1.77) |

| | | |
|---|---|---|
| LS mean difference vs OLM/HCTZ | −6.7 | −6.6 |
| (95% CI) | (−11.8, −1.6) | (−11.8, −1.5) |
| P-value | 0.010* | 0.011* |

Sex

| Male | | | | | | |
|---|---|---|---|---|---|---|
| n | 218 | | 191 | | 199 | |
| LS mean at Baseline (SE) (a) | 164.1 | (0.68) | 164.5 | (0.73) | 164.4 | (0.71) |
| LS mean Δ from BL (SE) | −40.8 | (1.01) | −42.2 | (1.08) | −35.9 | (1.06) |
| LS mean difference vs OLM/HCTZ | −4.9 | | −6.2 | | | |
| (95% CI) | (−7.8, −2.0) | | (−9.2, −3.3) | | | |
| P-value | <0.001* | | −0.001* | | | |
| Female | | | | | | |
| n | 126 | | 139 | | 155 | |
| LS mean at Baseline (SE) (a) | 166.1 | (0.91) | 165.7 | (0.86) | 164.9 | (0.82) |
| LS mean Δ from BL (SE) | −45.2 | (1.34) | −46.6 | (1.27) | −38.8 | (1.21) |

| | | |
|---|---|---|
| LS mean difference vs OLM/HCTZ | −6.4 | −7.8 |
| (95% CI) | (−10.0, −2.9) | (−11.3, −4.4) |
| P-value | <0.001* | <0.001* |

Race

| Caucasian | | | | | | |
|---|---|---|---|---|---|---|
| n | 254 | | 243 | | 259 | |
| LS mean at Baseline (SE) (a) | 164.0 | (0.64) | 164.5 | (0.65) | 164.2 | (0.63) |
| LS mean Δ from BL (SE) | −43.0 | (0.87) | −45.2 | (0.89) | −38.3 | (0.86) |
| LS mean difference vs OLM/HCTZ | −4.7 | | −7.0 | | | |
| (95% CI) | (−7.1, −2.3) | | (−9.4, −4.6) | | | |
| P-value | −0.001* | | −0.001* | | | |
| Black | | | | | | |
| n | 76 | | 72 | | 79 | |
| LS mean at Baseline (SE) (a) | 166.8 | (1.16) | 166.5 | (1.19) | 165.4 | (1.13) |
| LS mean Δ from BL (SE) | −40.1 | (1.99) | −40.0 | (2.04) | −33.9 | (1.95) |
| LS mean difference vs OLM/HCTZ | −6.2 | | −6.1 | | | |
| (95% CI) | (−11.7, −0.7) | | (−11.7, −0.5) | | | |
| P-value | −0.028* | | −0.031* | | | |
| Other | | | | | | |
| n | 15 | | 16 | | 17 | |
| LS mean at Baseline (SE) (a) | 169.4 | (2.33) | 167.3 | (2.26) | 166.6 | (2.19) |
| LS mean Δ from BL (SE) | −44.2 | (4.91) | −44.0 | (4.72) | −34.6 | (4.59) |

| | | |
|---|---|---|
| LS mean difference vs OLM/HCTZ | −9.7 | −9.4 |
| (95% CI) | (−23.3, 3.9) | (−22.7, 3.8) |
| P-value | 0.159 | 0.159 |

BMI

| <30 (kg/m²) | | | | | | |
|---|---|---|---|---|---|---|
| n | 160 | | 140 | | 163 | |
| LS mean at Baseline (SE) (a) | 164.4 | (0.82) | 166.6 | (0.87) | 164.0 | (0.81) |
| LS mean Δ from BL (SE) | −42.9 | (1.18) | −44.9 | (1.26) | −37.7 | (1.17) |
| LS mean difference vs OLM/HCTZ | −5.2 | | −7.2 | | | |
| (95% CI) | (−8.4, −1.9) | | (−10.6, −3.8) | | | |
| P-value | 0.002* | | <0.001* | | | |
| ≧30 (kg/m²) | | | | | | |
| n | 184 | | 190 | | 191 | |
| LS mean at Baseline (SE) (a) | 165.2 | (0.73) | 163.9 | (0.72) | 165.1 | (0.71) |
| LS mean Δ from BL (SE) | −42.1 | (1.12) | −43.4 | (1.10) | −36.6 | (1.10 |

| | | |
|---|---|---|
| LS mean difference vs OLM/HCTZ | −5.5 | −6.7 |
| (95% CI) | (−8.5, −2.4) | (−9.8, −3.7) |
| P-value | −0.001* | −0.001* |

Baseline Clinic SBP

| <163.3 mm Hg (b) | | | | | | |
|---|---|---|---|---|---|---|
| n | 173 | | 159 | | 175 | |
| LS mean at Baseline (SE) (a) | 157.7 | (0.46) | 157.1 | (0.48) | 157.1 | (0.46) |
| LS mean Δ from BL (SE) | −37.7 | (1.05) | −40.8 | (1.10) | −34.0 | (1.05) |
| LS mean difference vs OLM/HCTZ | −3.7 | | −6.9 | | | |
| (95% CI) | (−6.6, −0.8) | | (−9.8, −3.9) | | | |
| P-value | 0.013* | | <0.001* | | | |
| ≧163.3 mm Hg | | | | | | |
| n | 171 | | 171 | | 179 | |
| LS mean at Baseline (SE) (a) | 172.1 | (0.58) | 172.4 | (0.58) | 171.9 | (0.57) |
| LS mean Δ from BL (SE) | −47.1 | (1.23) | −47.3 | (1.23) | −40.2 | (1.20) |

| | | |
|---|---|---|
| LS mean difference vs OLM/HCTZ | −6.9 | −7.0 |
| (95% CI) | (−10.3, −3.5) | (−10.4, −3.6) |
| P-value | <0.001* | <0.001* |

eGFR

| Moderate Impairment 30 to <60 ml/min/1.73 m² | | | | | | |
|---|---|---|---|---|---|---|
| n | 25 | | 28 | | 23 | |
| LS mean at Baseline (SE) (a) | 166.7 | (2.23) | 165.8 | (2.11) | 167.5 | (2.32) |
| LS mean Δ from BL (SE) | −47.9 | (3.18) | −45.8 | (3.01) | −38.4 | (3.32) |
| LS mean difference vs OLM/HCTZ | −9.5 | | −7.4 | | | |
| (95% CI) | (−18.7, −0.3) | | (−16.4, 1.5) | | | |
| P-value | 0.042* | | 0.102 | | | |
| Mild Impairment 60 to <90 ml/min/1.73 m² | | | | | | |
| n | 217 | | 207 | | 240 | |
| LS mean at Baseline (SE) (a) | 164.9 | (0.70) | 164.8 | (0.72) | 164.5 | (0.66) |
| LS mean Δ from BL (SE) | −43.5 | (0.98) | −43.5 | (1.00) | −38.1 | (0.93) |
| LS mean difference vs OLM/HCTZ | −5.4 | | −5.4 | | | |
| (95% CI) | (−8.0, −2.7) | | (−8.1, −2.7) | | | |
| P-value | <0.001* | | <0.001* | | | |
| Normal ≧90 ml/min/1.73 m² | | | | | | |
| n | 102 | | 95 | | 90 | |
| LS mean at Baseline (SE) (a) | 164.3 | (0.93) | 165.2 | (0.96) | 164.5 | (0.99) |
| LS mean Δ from BL (SE) | −39.2 | (1.59) | −44.8 | (1.64) | −34.3 | (1.69) |

| | | |
|---|---|---|
| LS mean difference vs OLM/HCTZ | −4.9 | 10.4 |
| (95% CI) | (−9.4,− 0.3) | (−15.1, −5.8) |
| P-value | 0.037* | <0.001* |

Diabetes

| Yes | | | | | | |
|---|---|---|---|---|---|---|
| n | 53 | | 55 | | 61 | |
| LS mean at Baseline (SE) (a) | 166.5 | (1.59) | 165.1 | (1.56) | 165.3 | (1.49) |
| LS mean Δ from BL (SE) | −41.9 | (2.44) | −42.6 | (2.40) | −33.4 | (2.27) |
| LS mean difference vs OLM/HCTZ | −8.5 | | −9.2 | | | |
| (95% CI) | (−15.1, −1.9) | | (−15.8, −2.7) | | | |
| P-value | 0.012* | | 0.006* | | | |
| No | | | | | | |
| n | 291 | | 275 | | 293 | |
| LS mean at Baseline (SE) (a) | 164.5 | (0.57) | 165.0 | (0.59) | 164.5 | (0.57) |
| LS mean Δ from BL (SE) | −42.6 | (0.85) | −44.3 | (0.87) | −37.9 | (0.84) |

| | | |
|---|---|---|
| LS mean difference vs OLM/HCTZ | −4.7 | −6.4 |
| (95% CI) | (−7.0, −2.3) | (−8.8, −4.0) |
| P-value | <0.001* | <0.001* |

Hypertension Severity

| Grade 1 (SBP ≧140 to <160 mm Hg) | | | | | | |
|---|---|---|---|---|---|---|
| n | 72 | | 71 | | 93 | |
| LS mean at Baseline (SE) (a) | 153.8 | (0.61) | 153.3 | (0.61) | 153.9 | (0.53) |
| LS mean Δ from BL (SE) | −36.8 | (1.66) | −38.4 | (1.68) | −31.2 | (1.46) |
| LS mean difference vs OLM/HCTZ | −5.5 | | −7.2 | | | |
| (95% CI) | (−9.9, −1.2) | | (−11.6, −2.8) | | | |
| P-value | 0.013* | | 0.001* | | | |
| Grade 2 (SBP ≧160 to <180 mm Hg) | | | | | | |
| n | 238 | | 224 | | 228 | |
| LS mean at Baseline (SE) (a) | 165.9 | (0.35) | 166.4 | (0.37) | 166.6 | (0.36) |
| LS mean Δ from BL (SE) | −43.2 | (0.98) | −45.2 | (1.01) | −37.9 | (1.00) |
| LS mean difference vs OLM/HCTZ | −5.3 | | −7.3 | | | |
| (95% CI) | (−8.0,− 2.5) | | (−10.1, −4.5) | | | |
| P-value | <0.001* | | <0.001* | | | |
| Grade 3 (SBP ≧180 mm Hg) | | | | | | |
| n | 31 | | 32 | | 31 | |
| LS mean at Baseline (SE) (a) | 185.3 | (0.80) | 184.9 | (0.79) | 184.1 | (0.80) |
| LS mean Δ from BL (SE) | −53.1 | (3.03) | −52.8 | (2.97) | −47.6 | (3.03) |

| | | |
|---|---|---|
| LS mean difference vs OLM/HCTZ | −5.4 | −5.2 |
| (95% CI) | (−14.0, 3.1) | (−13.7, 3.2) |
| P-value | 0.208 | 0.223 |

Baseline Hypertension

| DBP <90 mm Hg | | | | | | |
|---|---|---|---|---|---|---|
| n | 85 | | 77 | | 102 | |
| LS mean at Baseline (SE) (a) | 162.5 | (1.11) | 160.4 | (1.17) | 161.0 | (1.01) |
| LS mean Δ from BL (SE) | −40.3 | (1.60) | −42.2 | (1.68) | −33.3 | (1.46) |
| LS mean difference vs OLM/HCTZ | −6.9 | | −8.8 | | | |
| (95% CI) | (−11.2, −2.6) | | (−13.2, −4.4) | | | |
| P-value | 0.002* | | <0.001* | | | |
| DBP ≧90 mm Hg | | | | | | |
| n | 259 | | 253 | | 252 | |
| LS mean at Baseline (SE) (a) | 165.6 | (0.61) | 166.4 | (0.61) | 166.1 | (0.62) |
| LS mean Δ from BL (SE) | −43.3 | (0.94) | −44.7 | (0.95) | −38.6 | (0.95) |

| | | |
|---|---|---|
| LS mean difference vs OLM/HCTZ | −4.7 | −6.1 |
| (95% CI) | (−7.3, −2.1) | (−8.8, −3.5) |
| P-value | <0.001* | <0.001* |

¹ Indicates statistically significant difference versus OLM/HCTZ at the 0.05 level.
Note:
Postbase1ine P-vales are obtained from an ANCOVA model with treatment as a factor and Baseline as a covariate.
(a) Baseline value is the last observation before the first dose of double-blind study drug.
(b) Median baseline SBP of all subjects.

In each subgroup, titration with TAK-491CLD led to large, clinically significant reductions in all blood pressure parameters, and for most of the subgroups, titration with either TAK-491CLD 40/25 or 80/25 mg led to statistically significantly greater decreases in clinic SBP from Baseline to Week 12 compared with OLM/HCTZ; exceptions that were not statistically significant between either TAK-491CLD titration group compared with the OLM/HCTZ titration group included subjects in the “Other” race subgroup and the Grade 3 hypertension groups, as well as subjects in the TAK-491CLD 80/25 mg titration group compared with the OLM/HCTZ titration group who had moderate renal impairment (GFR 30 to <60 ml/min/1.73 m²); however, SBP reductions were numerically greater with TAK-491CLD than with OLM/HCTZ in these relatively small subgroups. In each of these subgroups (ie, all 3 of these exceptions), the sample size totaled 32 or fewer subjects per titration group and reduced power to detect a statistical difference.

In a post hoc analysis, there was no statistical evidence (P>0.10) that response to an individual treatment was dependent on any one of the subgroups of age, sex, race, BMI, baseline hypertension severity, renal function, or diabetes.

### Safety Results:

In subjects treated for 12 weeks in 1 of 3 forced-titration treatment regimens (T AK-491CLD 40/25 mg titration [20/12.5→40/12.5→40/25 mg], TAK-491CLD 80/25 mg titration [40/12.5→80/12.5→80/25 mg], or OLM/HCTZ titration [20/12.5→40/12.5→40/25 mg]), the following was observed:
Subjects in the TAK-491CLD 40/25 and 80/25 mg titration groups had a higher incidence of TEAEs (approximately 71% each) compared with the OLM/HCTZ titration group (60.2%).
   The most common TEAEs (and overall frequencies) were blood creatinine increased (16.6%), dizziness (12.0%), fatigue (5.9%), and headache (5.4%).
   The incidence of blood creatinine increased was higher in the TAK-491CLD 40/25 and 80/25 mg titration groups (18.6% and 22.2%, respectively) compared with the OLM/HCTZ titration group (9.3%); a protocol-specified requirement to report adverse events for subjects with creatinine levels>30% from Baseline and >ULN at any visit may have contributed to these incidences.
   The incidence of fatigue was higher in the TAK-491CLD 40/25 mg titration group (9.3%) than in the TAK-491CLD 80/25 mg and OLM/HCTZ titration groups (4.0% and 4.4%, respectively).
   The incidence of dizziness was highest in the TAK-491CLD 80/25 mg titration group (16.5%) compared with TAK-491CLD 40/25 mg (11.5%) and the OLM/HCTZ titration group (8.0%).
   The incidence of hypotension (1.1% each) and syncope (0.6% and 0.8%) was low and similar in the TAK-491CLD 40/25 mg and OLM/HCTZ titration groups, respectively, and higher in the TAK-491CLD 80/25 mg titration group (4.3% for hypotension and 1.7% for syncope). The incidence of orthostatic hypotension, however, was similar between groups (range: 0.8% to 1.1%).
   A higher incidence of headache was reported in the OLM/HCTZ titration group (7.1%) compared with the TAK-491CLD 40/25 and 80/25 mg titration groups (5.4% and 3.7%, respectively).
The incidence of treatment-emergent SAEs was low and similar in the TAK-491CLD 80/25 mg and OLM/HCTZ titration groups (2.8% and 2.2%, respectively) and lowest in the TAK-491CLD 40/25 mg titration group (0.3%).
No deaths occurred during active treatment.
Increases of serum creatinine were observed in all titration groups, but increases ˜50% from Baseline and >ULN at any visit were more frequent in the TAK-491CLD 80/25 mg titration group (13.8%), than in the TAK-491CLD 40/25 mg titration group (8.6%) or the OLM/HCTZ titration group (5.6%).
   Most elevations of creatinine were transient, as indicated by lower frequencies of Final Visit and consecutive elevations than any visit elevations:
      At Final Visit, the incidence of creatinine increases ˜50% from Baseline and >ULN was relatively low and similar in the TAK-491CLD 40/25 mg and OLM/HCTZ titration groups (1.7% and 0.8%, respectively) but was higher in the TAK-491CLD 80/25 mg titration group (5.3%).
      The incidence of consecutive creatinine increases ˜50% from Baseline and >ULN was relatively low and similar in the TAK-491CLD 40/25 mg and OLM/HCTZ titration groups (1.4% and 2.8%, respectively) but was higher in the TAK-491CLD 80/25 mg titration group (4.4%).
   Nearly all subjects with elevated serum creatinine values at the Final Visit reversed to Baseline or Screening levels after discontinuation of study medication.
   Subjects with creatinine elevations experienced greater reductions in blood pressure than subjects without elevations.
Shifts from normal to low serum potassium and sodium were low and similar in all titration groups. The incidence of markedly low potassium (<3.0 mmol/L) levels was also low in all titration groups.
Mean increases in serum creatinine, blood urea nitrogen, and serum uric acid were more common in subjects who received TAK-491CLD than OLM/HCTZ. Small mean increases in serum glucose and triglycerides were observed in all titration groups at a similar frequency.
No clinically relevant changes were observed for physical examinations, ECG parameters, or vital signs, including heart rate, weight, and orthostasis.

### CONCLUSIONS

In this 12-week, randomized, double-blind, forced-titration blood pressure study comparing the efficacy and safety profiles of subjects with moderate to severe hypertension (SBP≧160 to ≦90 mm Hg) who were treated with TAK-491CLD 40/25 mg titration (20/12.5→40/12.5→40/25 mg), TAK-491CLD 80/25 mg titration (40/12.5→80/12.5→80/25 mg), or OLM/HCTZ titration (20/12.5→40/12.5→40/25 mg), the following was observed:
Both the TAK-491CLD 40/25 and 80/25 mg titration groups resulted in statistically significantly greater blood pressure reduction than the OLM/HCTZ titration group based on the primary endpoint of clinic SBP, indicating superior antihypertensive efficacy of TAK-491CLD.
The TAK-491CLD titration groups also resulted in significantly greater blood pressure reduction than OLM/HCTZ titration for the secondary endpoints of clinic DBP and 24-hour mean SBP, including each hourly interval of the ABPM analysis.
The safety and tolerability profile of the TAK-491CLD 40/25 mg and OLM/HCTZ titration groups were comparable, while the TAK-491CLD 80/25 mg titration group was associated with more frequent mechanism-based side effects, such as dizziness and reversible elevations of serum creatinine, and discontinuations due to adverse events.

All patents and publications cited herein are incorporated by reference into this application in their entirety.

The words “comprise”, “comprises”, and “comprising” are to be interpreted inclusively rather than exclusively.

What is claimed is:

1. A method of lowering blood pressure in a black patient in need of treatment thereof, the method comprising the steps of:
preferentially selecting a therapeutically effective amount of at least one angiotensin II receptor blocker (ARB) from a class of ARBs to lower the blood pressure in a black patient, wherein the ARB selected is azilsartan medoxomil; and
administering a combination of a therapeutically effective amount of the azilsartan medoxomil and a therapeutically effective amount of chlorthalidone to lower the blood pressure of the black patient.

2. The method of claim 1, wherein the patient is administered 20 mg once per day of azilsartan medoxomil.

3. The method of claim 1, wherein the patient is administered 40 mg once per day of azilsartan medoxomil.

4. The method of claim 1, wherein the patient is administered 80 mg once per day of azilsartan medoxomil.

5. The method of claim 1, wherein the chlorthalidone is administered simultaneously or sequentially with the azilsartan medoxomil.

6. The method of claim 1, wherein the chlorthalidone and azilsartan medoxomil are administered as separate dosage forms.

7. The method of claim 1, wherein the chlorthalidone and azilsartan medoxomil are administered in a single dosage form.

8. The method of claim 1, wherein the patient is administered 12.5 mg once per day of chlorthalidone.

9. The method of claim 1, wherein the patient is administered 25 mg once per day of chlorthalidone.

10. The method of claim 1, wherein the patient is administered 20 mg of azilsartan medoxomil and 25.0 mg of chlorthalidone once per day.

11. The method of claim 1, wherein the patient is administered 40 mg of azilsartan medoxomil and 25.0 mg of chlorthalidone once per day.

12. The method of claim 1, wherein the patient is administered 80 mg of azilsartan medoxomil and 25.0 mg of chlorthalidone once per day.

13. The method of claim 1, wherein the blood pressure is systolic or diastolic blood pressure.

14. The method of claim 1, wherein the patient suffers from hypertension, heart failure, stroke, chronic kidney disease, arrhythmia, peripheral artery disease, coronary artery disease or combinations thereof.

15. The method of claim 1, wherein the hypertension is stage 1 primary hypertension.

16. The method of claim 1, wherein the hypertension is stage 2 primary hypertension.

17. A method of lowering blood pressure in a black patient in need of treatment thereof, the method comprising the steps of:
preferentially administering a combination of a therapeutically effective amount of at least one angiotensin II receptor blocker (ARB) from a class of ARBs and a therapeutically effective amount of chlorthalidone to lower the blood pressure in a black patient, wherein the ARB administered to the black patient is azilsartan medoxomil.

18. The method of claim 17, wherein the patient is administered 20 mg once per day of azilsartan medoxomil.

19. The method of claim 17, wherein the patient is administered 40 mg once per day of azilsartan medoxomil.

20. The method of claim 17, wherein the patient is administered 80 mg once per day of azilsartan medoxomil.

21. The method of claim 17, wherein the chlorthalidone is administered simultaneously or sequentially with the azilsartan medoxomil.

22. The method of claim 17, wherein the chlorthalidone and azilsartan medoxomil are administered as separate dosage forms.

23. The method of claim 17, wherein the chlorthalidone and azilsartan medoxomil are administered in a single dosage form.

24. The method of claim 17, wherein the patient is administered 12.5 mg once per day of chlorthalidone.

25. The method of claim 17, wherein the patient is administered 25 mg once per day of chlorthalidone.

26. The method of claim 17, wherein the patient is administered 20 mg of azilsartan medoxomil and 25.0 mg of chlorthalidone once per day.

27. The method of claim 17, wherein the patient is administered 40 mg of azilsartan medoxomil and 25.0 mg of chlorthalidone once per day.

28. The method of claim 17, wherein the patient is administered 80 mg of azilsartan medoxomil and 25.0 mg of chlorthalidone once per day.

29. The method of claim 17, wherein the blood pressure is systolic blood pressure.

30. The method of claim 17, wherein the patient suffers from hypertension, heart failure, stroke, chronic kidney disease, arrhythmia, peripheral artery disease, coronary artery disease or combinations thereof.

31. The method of claim 17, wherein the hypertension is stage 1 primary systolic hypertension.

32. The method of claim 17, wherein the hypertension is stage 2 primary systolic hypertension.

33. The method of claim 1, wherein the patient is administered 20 mg of azilsartan medoxomil and 12.5 mg of chlorthalidone once per day.

33. The method of claim 1, wherein the patient is administered 40 mg of azilsartan medoxomil and 12.5 mg of chlorthalidone once per day.

34. The method of claim 1, wherein the patient is administered 80 mg of azilsartan medoxomil and 12.5 mg of chlorthalidone once per day.

35. The method of claim 17, wherein the patient is administered 20 mg of azilsartan medoxomil and 12.5 mg of chlorthalidone once per day.

36. The method of claim 17, wherein the patient is administered 40 mg of azilsartan medoxomil and 12.5 mg of chlorthalidone once per day.

37. The method of claim 17, wherein the patient is administered 80 mg of azilsartan medoxomil and 12.5 mg of chlorthalidone once per day.
